(19) United States
(12) Patent Application Publication
(10) Pub. No.: US 2022/0175954 A1
(43) Pub. Date: Jun. 9, 2022

(54) HYPOXIA-INDUCING CRYOGELS

(71) Applicant: Northeastern University, Boston, MA (US)

(72) Inventors: Sidi A. Bencherif, Boston, MA (US); Thibault Colombani, Boston, MA (US); Zachary Rogers, Brookline, MA (US)

(21) Appl. No.: 17/600,198

(22) PCT Filed: Apr. 2, 2020
(86) PCT No.: PCT/US20/26310

§ 371 Date: Sep. 30, 2021

Related U.S. Application Data
(60) Provisional application No. 62/828,110, filed on Apr. 2, 2019.

(51) Int. Cl. A61K 47/69; A61K 38/08; A61K 47/58

(52) U.S. Cl. CPC A61K 47/6903; A61K 47/58; A61K 38/08

(57) ABSTRACT

The present disclosure relates to a hypoxia-inducing cryogel, comprising one or more polymer and one or more hypoxia-inducing agent. The present disclosure additionally relates to a hypoxia-inducing construct, comprising a cryogel and a support. Methods of reducing concentration of oxygen in a medium, comprising contacting the medium with a hypoxia-inducing cryogel (HIC) or a hypoxia-inducing construct are disclosed. Additionally, methods of inducing hypoxia in a cell, comprising contacting the cell with a medium, wherein the medium comprises a HIC or a hypoxia-inducing construct are disclosed.

### RELATED APPLICATIONS

This application claims the benefit of priority to U.S. Provisional Patent Application No. 62/828,110, filed Apr. 2, 2019.

### BACKGROUND

Hypoxia, defined as low oxygen tension, is often a pathological condition due to a deprivation of adequate oxygen supply at the tissue level. Cellular responses to hypoxia are primarily induced by hypoxia-inducible factors (HIFs). HIFs are transcription factors that respond to decreases in available oxygen in the cellular environment, or hypoxia. HIFs, stabilized in hypoxic conditions, play a crucial role in adaptive cell responses to low oxygen tensions through transcriptional activation of over 100 downstream genes involved in vital biological processes. For example, HIFs act as key regulators of the glucose metabolism, angiogenesis, immune suppression, resistance to apoptosis and autophagy, stem cell phenotype, but also cell division, migration and invasion.

Hypoxia has been associated with a number of diseases (obesity, cancer, coronary artery disease, atherosclerosis, fatty liver disease, stroke, etc.), healthy human tissues (brain, skin, muscle, eye, bone marrow, etc.), and regulation of immunological processes (immunosuppression, inflammation, etc.) and currently a major research interest. More particularly, hypoxia is a physiological state in some tissues (such as cartilages, endothelium, mucosa) or during several biological events (such as embryogenesis, tissue regeneration). In hypoxia, cell metabolism and physiological functions are deeply changed, impacting the cell phenotype and behavior. For instance, hypoxia is a characteristic feature of solid tumors and results in their metabolic adaptation leading to tumor cell growth and invasion, resistance to apoptosis, and multi-drug resistance. Thus, hypoxic cell culture conditions are desirable for basic research, disease modeling, drug screening, regenerative medicine and several other fields of research. However, in cell cultures oxygen concentrations are usually not controlled. Although a decrease in oxygen concentration is the optimal hypoxia model, the problem faced by many researchers is access to a hypoxia chamber or a CO₂ incubator with regulated oxygen levels, which is not possible in many research laboratories. Furthermore, current technologies for maintaining hypoxic cell cultures are lacking. For instance, chemically-induced hypoxia (e.g., cobalt chloride-induced hypoxia) does not accurately recapitulate hypoxia because it does not induce all the crucial hypoxia-associated pathways. Portable chambers, which are equilibrated to hypoxic conditions and then placed in a conventional cell culture chamber, prevent scientists from manipulating or analyzing their cells (which happens often in the cell culture process) without disturbing hypoxia. Tri-gas incubators (i.e., hypoxic incubators), which overlay cells with nitrogen to control the oxygen concentration, suffer from the same challenges as the portable chambers, but are also costly, requiring a constant source of nitrogen. Lastly, hypoxic workstations, which allow for cell culture, handling and analysis simultaneously, are expensive (>$100,000), constrain scientists to a small working area and are limiting in which analyses can be completed.

There is a need for advanced biomaterials that can induce hypoxic conditions, for example: (i) to emulate tissues with reduced oxygen tensions (e.g. bone, cartilage, brain), (ii) to create more reliable tumor models in vitro and in vivo, (iii) to investigate immune cell behavior in a hypoxic tumor-like microenvironment, (iv) to use hypoxia in biomaterial-based vaccines for autoimmune diseases, and/or (v) to preserve primary cell phenotype.

Hydrogels have been used for a number of biomedical applications because of their three-dimensional (3D) nature, high water content and wide range of polymers that can be used for their fabrication. Hydrogels have recently gained momentum because they can mimic key features of the extracellular matrix (ECM), mainly due to their structural similarity with native tissues and their tunable biophysical properties.

Recent advances in hydrogel fabrication led to the development of cryogels, highly macroporous hydrogel scaffolds. Cryogels are synthesized by cryogelation of monomers or polymeric precursors at subzero temperatures. The procedure of cryogelation occurs through the following steps: phase separation with the ice crystal formations, cross-linking, and polymerization followed by thawing of the ice crystals forming an interconnected porous cryogel network. These cryogels can have a high level of biocompatibility and display biomechanical properties that recapitulate temporal and spatial complexity of soft native tissues. Advantages of cryogels include an exceptional combination of highly porous characteristics with sufficient osmotic stability and mechanical strength. As a result, they have been extensively used for a variety of biomedical uses. Another essential feature of cryogels is the simple approach through which cryogels are synthesized and the application of aqueous solvent(s) making these fit for the diverse biological and biomedical applications. Different modifications of these cryogel systems have been sought, which entails coupling of a variety of ligands to its surfaces, grafting of the polymeric chains to the surface of cryogels or IPN of two or more polymers to develop a cryogel for diverse applications. For instance, cryogels can be functionalized with proteins and/or peptides to enable biological activities (e.g. cell adhesion ligands, antibodies, enzymes), can encapsulate bioactive molecules and control the spatiotemporal release (e.g. cytokines, growth factors), and can be biodegradable (e.g. proteolytic or hydrolytic degradability, oxidation). Finally, cryogels can be delivered in a minimally invasive manner via syringe injection through a conventional small-bore needle, removing the need for surgical implantations and associated side effects.

### SUMMARY

Compositions and materials described herein can serve, for example, as hypoxic 3D microenvironments to study the impact of hypoxia on (a) tumor development, progression, aggressiveness and resistance to therapeutics, (b) on primary cell differentiation and phenotype, (c) on immune cell migration, and function, and (d) on immune responses. The present disclosure also relates to hypoxia-inducing cryogels (HIC) devices for two-dimensional (2D) hypoxic cell culture, labeled as HIC_{2D}, that can be directly added to cell cultures to create hypoxic conditions (FIG. 1). HIC_{2D} is a technology that can maintain hypoxic cell culture conditions under atmospheric oxygen without locking cells in an environment in which oxygen tension is controlled. Most importantly, HIC_{2D} can allow scientists to simultaneously maintain hypoxia and perform hassle-free cell culture procedures and analyses in a laboratory setting under ambient air.

The compositions and materials can also be used alone in various media as a system to efficiently deplete oxygen without any toxic byproducts. As such, embodiments of the compositions and materials can be a substitute of current bulky, and/or expensive, and/or inflexible systems used to induce hypoxic conditions, such as hypoxic chambers, hypoxic incubators (e.g., tri-gas incubators), or hypoxic cabinets and low oxygen workstations. Embodiments of the compositions and materials can also be adapted to known cell culture methods, therefore being used as a flexible tool to induce hypoxia.

In some embodiments, the present disclosure relates to a hypoxia-inducing cryogel, comprising one or more polymer and one or more hypoxia-inducing agent.

In some embodiments, the present disclosure relates to a hypoxia-inducing construct, comprising a cryogel and a support.

In some embodiments, the present disclosure relates to a method of reducing concentration of oxygen in a medium, comprising contacting the medium with a hypoxia-inducing cryogel (HIC) or a hypoxia-inducing construct.

In some embodiments, the present disclosure relates to a method of inducing hypoxia, comprising contacting the cell with a medium, wherein the medium comprises a HIC or a hypoxia-inducing construct.

In some embodiments, the present disclosure relates to a method of inducing hypoxia, comprising contacting the cell with a HIC or a hypoxia-inducing construct.

### BRIEF DESCRIPTION OF THE DRAWINGS

FIG. 1 shows a schematic representation of the HIC_{2D} manufacturing process, mechanism of action, and design. HICs_{2D} are fabricated by cryopolymerization and rapidly deplete oxygen in cell culture media. (1) Monomer/polymers (e.g., HAGM) are mixed with an initiator system, acrylate-PEG-glucose oxidase (APG) and acrylate-PEG-catalase (APC) in water. The mixture is subsequently transferred to a mold and incubated at subzero temperature (−20° C.). (2) Ice crystals form, concentrating the HAGM and initiator in a non-frozen liquid phase where cross-linking occurs. (3) Molds are brought to room temperature, melting ice crystals and leaving behind a macroporous cryogel, referred as HIC_{2D}. (4) HIC_{2D} is added into a well plate containing cell culture media, where APG converts water (H₂O), D-glucose (G) and oxygen (O₂) to hydrogen peroxide (H₂O₂) and D-glucono-δ-lactone (GL), producing a hypoxic milieu. APC quickly breaks down H₂O₂, maintaining a cell-friendly environment (nontoxic). Right side: photographs of HIC_{2D} in PBS in a single well of a 24-well plate. HICs_{2D} were designed to float on top of the cell culture medium, preventing interference with cells (e.g., B16-F10 melanoma cells) growing on the bottom of the plate.

FIG. 2 shows a schematic representation of HIC_{2D} in a single well of a 24-well plate. When immersed in cell culture media, HIC_{2D} depletes oxygen quickly (<1 h) and induce cellular hypoxia.

FIG. 3 shows confocal images depicting the interconnected microporous structure of HICs and blank cryogels.

FIG. 4 shows a chart demonstrating pore size distribution of HICs and blank cryogels. Values represent mean and standard error of the mean (SEM) (n=6).

FIG. 5 shows a chart demonstrating pore connectivity of HICs and blank cryogels. Values represent mean and standard error of the mean (n=6).

FIG. 6 shows a chart demonstrating mass swelling ratio of HICs and blank cryogels. Values represent mean and standard error of the mean (n=6).

FIG. 7 shows a chart demonstrating Young's moduli of HICs and blank cryogels. Values represent mean and standard error of the mean (n=6).

FIG. 8 shows microphotographs of HICs and blank cryogels before and after injection (representative of n=10 samples).

FIG. 9 demonstrates controlled and sustainable oxygen depletion by HIC_{2D} over 3 hours. Cryogel rings (˜200 μL), either HICs_{2D} or blank cryogels, were placed in wells of a 24-well plate containing 2 mL of DMEM media/well supplemented with excess D-glucose (50 g/L). The cryogels floated to the top of the medium. Oxygen-measuring probes were inserted into the bottom of the wells and oxygen concentration was monitored.

FIG. 10 demonstrates controlled and sustainable oxygen depletion by HIC_{2D} over 24 hours. Cryogel rings (˜200 μL), either HICs_{2D} or blank cryogels, were placed in wells of a 24-well plate containing 2 mL of DMEM media/well supplemented with excess D-glucose (50 g/L). The cryogels floated to the top of the medium. Oxygen-measuring probes were inserted into the bottom of the wells and oxygen concentration was monitored.

FIG. 11 demonstrates comparison between oxygen depletion kinetics of a 24-well plate containing HIC_{2D} incubated in a standard incubator (blue) vs. HIC_{2D}-free well plate incubated in a hypoxic incubator (green). Well loading: 2 mL of DMEM+4.5 g/L D-glucose media/well. Once hypoxia was reached (<5% O₂), the well plates were removed from the regular incubator to examine the rate of equilibration to normoxia when stored under atmospheric oxygen.

FIG. 12 shows a plot demonstrating oxygen depletion from blank cryogels or HICs in normoxia for 11 days.

FIG. 13 shows a plot demonstrating oxygen concentration over 48 h in DMEM media supplemented with 4.5 g/L of D-glucose.

FIG. 14 shows a plot demonstrating oxygen concentration over 48 h in DMEM media before and after addition of 4.5 g/L of D-glucose.

FIG. 15 shows a plot demonstrating HIC-mediated glucose consumption over time in DMEM media containing 4.5 g/L of D-glucose.

FIG. 16 shows a plot demonstrating H₂O₂ release after 24 h from HICs containing both APG and APC, or lacking one enzyme (APC or APG), in DMEM media containing 4.5 g/L of D-glucose. Blank cryogels were used a control group. Values represent mean and standard error of the mean (n=4). Data were analyzed using one-way analysis of variance (ANOVA) and Dunnett post-test (comparison to APC-free HICs), *p<0.05.

FIG. 17 demonstrates cellular hypoxia induced by HIC_{2D}. HIC_{2D}-mediated cellular hypoxia was qualitatively analyzed by confocal microscopy. Blank cryogels were used a control group. B16-F10 melanoma cells were stained with Image-iT® Red, a reversible fluorescent marker that detects cellular hypoxia (pink) below 5%. The images are representative of n=3 samples per conditions.

FIG. 18 demonstrates cellular hypoxia induced by HIC_{2D}. HIC_{2D}-mediated cellular hypoxia was quantitatively analyzed by data processing. Blank cryogels were used a control group. B16-F10 melanoma cells were stained with Image-iT® Red, a reversible fluorescent marker that detects cellular hypoxia (pink) below 5%. Cellular hypoxia was quantified with ImageJ® software (i.e., % of number of fluorescently labeled cells over total cell count). Values represent mean and standard error of the mean (n=3). Data were analyzed using unpaired two-tailed t-test (Mann-Whitney), *p<0.05.

FIG. 19 shows B16-F10 melanoma cell viability after 24 h incubation within HICs or blank cryogels in normoxic conditions. The images are representative of n=6 samples per conditions. Staining: “Blue”=nuclei stained with DAPI, “red”=dead cells stained with ViaQuant Far Red, “green”=actin cytoskeleton stained with alexa fluor 488 phalloidin, “yellow”=polymer walls stained with rhodamine, purple=hypoxic cells stained with hypoxyprobe.

FIG. 20 shows a chart demonstrating B16-F10 melanoma cell viability after 24 h incubation within HICs or blank cryogels in normoxic conditions. Values represent mean and standard error of the mean (n=6). Data were analyzed using unpaired two-tailed t-test (Mann-Whitney), *p<0.05.

FIG. 21 shows confocal images of hypoxic B16-F10 cells after 24 h of incubation within HICs or blank cryogels under normoxic conditions. The images are representative of n=6 samples per conditions. Staining: “Blue”=nuclei stained with DAPI, “purple”=hypoxic cells stained with hypoxyprobe.

FIG. 22 shows a chart demonstrating B16-F10 cell hypoxia quantification after 24 h incubation within HICs or blank cryogels in normoxic conditions. Values represent mean and standard error of the mean (n=6). Data were analyzed using unpaired two-tailed t-test (Mann-Whitney), **p<0.01.

FIG. 23 shows charts demonstrating HICs-induced changes in levels of biomarkers, resulting the change of cancer cell phenotype. Evaluation of 4T1 breast cancer cell gene expression after 24 h or 48 h incubation within HICs or blank cryogels under normoxic conditions. Values represent mean and standard error of the mean (n=6). Data were analyzed using one-way analysis of variance (ANOVA) and Dunnett post-test (comparison to HICs), *p<0.05.**p<0.01, ***p<0.001.

FIG. 24 shows a cell viability plot and the half maximal inhibitory concentration (IC50) of 4T1 breast cancer cells (100,000 cells) when cultured in blank cryogels and exposed to various doxorubicin concentrations (0-100 μM) after 24, 48 and 72 h of incubation. IC50 was calculated using a linear regression in the linear part of each curve (Y=mX+n with Y=50 and X=IC50). Data are representative of n=6 samples per set of conditions.

FIG. 25 shows confocal microscopy images depicting cell viability of 4T1 breast cancer cells (100,000 cells) when cultured in blank cryogels or HICs and exposed to various doxorubicin concentrations (0 or 2×10³ nM) for 72 h of incubation. The images are representative of n=6 samples per set of conditions.

FIG. 26 shows a cell viability plot and the IC50 of B16-F10 melanoma cells (100,000 cells) when cultured in blank cryogels and exposed to various doxorubicin concentrations (0-100 μM) for 24, 48 and 72 h of incubation. Data are representative of n=6 samples per set of conditions.

FIG. 27 shows confocal microscopy images depicting cell viability of B16-F10 melanoma cells (100,000 cells) when cultured in blank cryogels or HICs and exposed to various doxorubicin concentrations (0 or 2×10³ nM) for 72 h of incubation. The images are representative of n=6 samples per set of conditions.

FIG. 28 shows a cell viability plot and the IC50 of 4T1 breast cancer cells (100,000 cells) when cultured in HICs and exposed to various doxorubicin concentrations (0-100 μM) for 24, 48 and 72 h of incubation. Data are representative of n=6 samples per set of conditions.

FIG. 29 shows a cell viability plot and the IC50 of B16-F10 melanoma cells (100,000 cells) when cultured in HICs and exposed to various doxorubicin concentrations (0-100 μM) for 24, 48 and 72 h of incubation. Data are representative of n=6 samples per set of conditions.

FIG. 30 shows a cell viability plot and the IC50 of 4T1 breast cancer cells (100,000 cells) when cultured in blank cryogels and exposed to various cisplatin concentrations (0-100 μM) for 24, 48 and 72 h of incubation. Data are representative of n=6 samples per set of conditions.

FIG. 31 shows a cell viability plot and the IC50 of 4T1 breast cancer cells (100,000 cells) when cultured in HICs and exposed to various cisplatin concentrations (0-100 μM) for 24, 48 and 72 h of incubation. Data are representative of n=6 samples per set of conditions.

FIG. 32 shows a cell viability plot and the IC50 of B16-F10 melanoma cells (100,000 cells) when cultured in blank cryogels and exposed to various cisplatin concentrations (0-100 μM) for 24, 48 and 72 h of incubation. Data are representative of n=6 samples per set of conditions.

FIG. 33 shows a cell viability plot and the IC50 of B16-F10 melanoma cells (100,000 cells) when cultured in HICs and exposed to various cisplatin concentrations (0-100 μM) for 24, 48 and 72 h of incubation. Data are representative of n=6 samples per set of conditions.

### DETAILED DESCRIPTION

Ice-templated cryogels are a class of materials with a highly porous interconnected structure that are produced using a cryotropic gelation (or cryogelation) technique. Cryogelation is a technique in which the polymerization-crosslinking reactions are conducted in quasi-frozen reaction solution. During freezing of the monomer(s) solution, the monomer(s) and the initiator system are expelled from the ice concentrate within the channels between the ice crystals, so that the reactions only take place in these unfrozen liquid channels. After polymerization and, after melting of ice, a porous material is produced whose microstructure is a negative replica of the formed ice. Ice crystals act as porogens. Pore size is tuned by altering the temperature of the cryogelation process. For example, the cryogelation process is typically carried out by quickly freezing the solution at −20° C. Lowering the temperature to, e.g., −80° C., would result in more ice crystals and smaller pores. Methods for immobilizing enzymes on polymers are disclosed, for example, in U.S. Pat. Nos. 10,045,947, 9,675,561, 8,975,309, 8,569,062, and 7,547,395, each of which is incorporated herein by reference in its entirety.

Hypoxia-inducing cryogels (HICs) are cryogels comprising hypoxia-inducing agents, which can be covalently or non-covalently attached to the polymer constituents of the hydrogel. As used herein, the term “hypoxia-inducing agent” refers to any agent, species, or moiety that can reduce the concentration of O₂ in its environment. Hypoxia-inducing agents can reduce oxygen concentrations by undergoing a chemical reaction with oxygen, by catalyzing a chemical reaction that consumes oxygen, or by physically or chemically sequestering oxygen from the environment. In some embodiments, enzymes glucose oxidase (GOX) and catalase (CAT) enzymes can be used as hypoxia-inducing agents. Additionally, or alternatively, other agents such as ferulic acid (C₁₀H₁₀O₄) and various oxidase enzymes (e.g., NAPDH oxidase, laccase, monoamine oxidase, etc.) can be utilized as hypoxia-inducing agents. In some embodiments, the HICs comprise acrylate-PEG-glucose oxidase (APG) and/or acrylate-PEG-catalase (APC).

Enzyme immobilization can be accomplished through physical adsorption/entrapment, electrostatic forces, covalent crosslinking, or biomolecule binding. Methods for immobilizing enzymes on polymers are disclosed, for example, in U.S. Pat. Nos. 8,889,373, 8,561,811, 8,440,441, 6,858,403, and 4,556,554, and U.S. Patent Application Publications Nos. 2005/0127002 and 2011/0117596, each of which is incorporated herein by reference in its entirety. A method of covalently attaching proteins to acrylate-PEG polymer is described, for example, in U.S. Pat. No. 8,481,073, which is incorporated herein by reference in its entirety.

HICs constitute a powerful platform to efficiently deplete oxygen in medium or solutions containing D-glucose. Alone, HICs could be used as a conditioner to remove oxygen from solutions, but also as a tool to induce hypoxic conditions in several biological systems already used in research.

HICs can be combined with cells in order to develop advanced 3D-tissue models. For cancer modeling, HICs can be used to understand the tumor development in a hypoxic environment. HICs can also be used to generate cancer cells with more aggressive phenotypes, for anti-cancer drug screening, for cancer-immune cell interaction, and to study cancer-driven immunosuppression. HICs could also be used to develop more representative in vivo animal models, by generating tumor cells in vitro with a metastatic phenotype, or by being injected with cancer cells as support for in vivo tumor formation.

HICs could also be used for the development of vaccines. As HICs can develop immunopermissive environments and can be loaded with biomolecules, HICs could be used to form in vivo tolerogenic immune cells, acting therefore as an auto-immune vaccine.

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification. The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, the term “biocompatible” refers to materials that are, with any metabolites or degradation products thereof, generally non-toxic and cause no significant adverse effects to living cells and tissues.

### Exemplary Features

In some embodiments, the present disclosure relates to large-size macroporous and biodegradable cryogels as a hypoxic 3D platform that can be administered via non-invasive strategies.

In some embodiments, the present disclosure relates to utilizing biocompatible polymers or monomers undergoing cryopolymerization. Suitable polymers and monomers include naturally derived polymers (peptides, proteins, nucleic acids, such as DNA strands, deoxyribonucleotide monomers, GRGDS peptide; alginate, hyaluronic acid, chitosan, heparin, carboxymethyl cellulose, cellulose, elastin, gelatin, carob gum, collagen, laminin, fibronectin, etc.) and semi-synthetic and synthetic polymers and copolymers, such as (poly(ethylene glycol) (PEG), pegylated proteins, pegylated polysaccharides, acrylate-PEG, PEG-co-poly(glycolic acid; PGA), PEG-co-poly(L-lactide; PLA), poly(2-hydroxyethyl methacrylate) (pHEMA), poly-2-hydroxyethylacrylate (polyHEA), polyacrylamide (PAAm), poly(N-isopropylacrylamide) (PNIPAAm), etc.). Semi-synthetic polymers are natural polymers (such as peptides, proteins, glycoproteins, lipids, nucleic acids, and polysaccharides) grafted with different synthetic substituents, including small molecules and/or other polymers.

In some embodiments, the present disclosure relates to minimally invasive delivery of compositions and materials described herein, for example, preformed biomaterials.

In some embodiment, in addition to the free radical polymerization process to cross-link the polymers and make chemically cross-linked injectable cryogels (polymerization time is about 17 hr), gels described herein can be polymerized using other processes. Injectable cryogels can be classified under two main groups according to the nature of their cross-linking mechanism, namely chemically and physically cross-linked gels. Covalent cross-linking processes include, but are not limited to, radical polymerization (vinyl monomers reaction), Michael-type addition reaction (vinyl-thiol reaction), polycondensation (esterification reaction between alcohols and carboxylic acids or amide formation between carboxylic acids and amines), oxidation (thiol-thiol cross-linking), thiol-maleimide “click” chemistry, aldehyde-mediated reactions, click chemistry (1,3-dipolar cycloaddition of organic azides and alkynes), Diels-Alder reaction (cycloaddition of dienes and dienophiles), oxime, imine and hydrazone chemistries. Non-covalent cross-linking include, but are not limited to, ionic cross-linking (e.g. alginate crosslinking with calcium, magnesium, potassium, barium), self-assembly (phase transition in response to external stimuli, such as temperature, pH, ion concentration, hydrophobic interactions, light, metabolite, and electric current).

In some embodiments, the disclosed cryogels are oxygen depleting.

In some embodiments, the disclosed cryogels are preformed hypoxia-inducing cryogels. In some embodiments, the disclosed macroporous scaffolds for 2D or 3D cell culture are depleting oxygen/ inducing hypoxic conditions in a controlled and sustained fashion.

In some embodiments, the compositions and materials disclosed herein induce local hypoxic environments.

In some embodiments, the compositions and materials disclosed herein allow immunosuppression of immune cells under normal or physiological oxygen tension.

In some embodiments, the compositions and materials disclosed herein allow promotion of immune cell regulatory function and activity.

In some embodiments, the compositions and materials disclosed herein allow induction of angiogenesis.

In some embodiments, the compositions and materials disclosed herein allow induction of stemness cell phenotype.

In some embodiments, the compositions and materials disclosed herein provide antibacterial activity.

In some embodiments, the compositions and materials disclosed herein can be used as part of an injectable system for controlled delivery of biomolecules (e.g., cytokines, adjuvants, immunosuppressors, or checkpoint inhibitor)

In some embodiments, lyoprotectants (e.g., trehalose, sucrose, glucose, etc.) can be used to enhance the efficacy of oxygen depletion after chemical modification of cryopolymerization.

In some embodiments, cryogels can be optionally loaded with bioactive molecules depending on the application. For example, cytokines, adjuvants or checkpoint inhibitors can be used to promote the differentiation of immune cells into regulatory cells or cell promoting tissue regeneration. For example, chemokines can be encapsulated to study cell migration in hypoxia, and growth factors can be loaded to investigate cell differentiation in hypoxic conditions.

Exemplary Advantages and Improvements over Existing Methods, Devices, or Materials

(1) Compositions and materials allow cost efficiency and reproducibility across laboratories.

(2) Chemical modification of enzymes (e.g., glucose oxidase (GOX), catalase (CAT), etc.) to allow their grafting within cryogels.

(3) Covalent grafting of enzymes to cryogels allow them to be removed at any point during a cell culture process, allowing scientists to control whether their cultures are hypoxic temporally. Additionally, the grafting of enzymes enhances their stability, prolonging the duration in which they are active.

(4) Grafting of GOX, CAT, or other oxygen-depleting molecules (e.g., ferulic acid, etc.) to cryogels to deplete oxygen in a controllable and sustained fashion.

(5) Interconnected macroporous network, which increases the mass transfer rates of substrates (e.g., D-glucose, oxygen, and hydrogen peroxide) to the enzymes compared to nanoporous (i.e., mesoporous) scaffolds such as hydrogels.

(6) HICs and HIC_{2D} can be synthesized with any shape, volume or surface area, allowing the technology to be adapted to any cell culture system (e.g., well plates, T-flasks, Petri dishes, etc.).

(7) HICs with shape memory properties.

(8) HICs injectable through conventional small-bore needles.

(9) HICs with antimicrobial properties.

(10) HICs and HICs_{2D} are hypoxia-inducing technologies that can maintain hypoxia in ambient conditions (21% O₂), allowing scientists to execute all cell culture handling and analysis procedures while maintaining hypoxia.

(11) HICs and HICs_{2D} reach hypoxia within an hour, whereas commercial technologies require several hours to reach hypoxia.

(12) Other than chemically-induced hypoxia (e.g., cobalt chloride-induced chemical hypoxia), which does not accurately recapitulate hypoxia, HICs and HIC_{2D} are the only laboratory consumable products that induce hypoxia, obviating the need for large and bulky equipment, gas use (expensive and environmentally unfriendly), and maintenance.

(13) HICs and HIC_{2D} are low cost and user-friendly.

### Exemplary Commercial Applications

(1) Compositions and materials described herein, for example, injectable macroscopic nanocomposite biomaterials, can be useful as surgical tissue adhesives, space-filling injectable materials for hard and soft tissue repair, drug delivery, and tissue engineering.

(2) Compositions and materials described herein can be used, for example, for tissue engineering, for tissue repair, in media conditioner, for in vitro hypoxia modeling, for immunoengineering, for auto-immune therapy, for wound healing, as biosensors, in wine production, as anti-microbial systems, as food and beverage additive, in biofuel cells, for oxygen depletion, and in bioreactors.

(3) Compositions and materials disclosed herein can be used to create hypoxic cell culture conditions in well plates (e.g., 4-well, 12-well, 24-well, 48-well, 96-well, etc.), dishes (e.g., 35×10 mm, 100×21 mm, etc.) and flasks (e.g., 25 cm² to 225 cm²).

(4) Compositions and materials disclosed herein can be used to create hypoxic cell culture conditions for cancer cells, organoids, stem cells, anaerobic bacteria (Bacteroides, Prevotella, Clostridium, etc.), healthy human tissues (e.g., brain, bone, cartilage, etc.), diseased human tissues (e.g., tumors), etc.

(5) Compositions and materials disclosed herein can be used to perform oxygen-sensitive chemical reactions (e.g., polymerization).

(6) Compositions and materials disclosed herein can be used as an alternative to oxygen-scavenging agents, which are often toxic and harmful to the environment, in applications such as food preservation, transportation of microbiological samples, tissue engineering (e.g., dopamine-containing biomaterials), etc.

In some embodiments, the present disclosure relates to a hypoxia-inducing cryogel, comprising one or more polymers and one or more hypoxia-inducing agents.

In some embodiments, the one or more polymers are biocompatible.

In some embodiments, the one or more polymers are hydrophilic.

In some embodiments, the one or more polymers are independently selected from the group consisting of DNA strands, peptides, proteins, alginate, hyaluronic acid, chitosan, heparin, carboxymethyl cellulose, cellulose, carob gum, hyaluronic acid glycidyl methacrylate (HAGM), methacrylated gelatin, methacrylated alginate, poly(ethylene glycol) (PEG), acrylate-PEG, methacrylate-PEG, PEG-co-poly(glycolic acid), PEG-co-poly(L-lactide), poly(2-hydroxyethyl methacrylate) (pHEMA), poly-2-hydroxyethylacrylate (polyHEA), polyacrylamide (PAAm), and poly(N-isopropylacrylamide) (PNIPAAm), and copolymers and combinations thereof.

In some embodiments, the one or more polymers comprise HAGM.

In some embodiments, the one or more polymers comprise acrylate-PEG or methacrylate-PEG.

In some embodiments, the one or more polymers comprise a peptide or a protein selected from the group consisting of a synthetic peptide, elastin, gelatin, collagen, laminin, fibrin, fibrinogen, vitronectin, fibronectin, and a selectin.

In some embodiments, the synthetic peptide is selected from the group consisting of GRGDS, GGGGRGDSP, and GFOGER.

In some embodiments, the peptide or the protein is covalently attached to at least one polymer of the one or more polymers.

In some embodiments, the peptide or the protein is covalently attached to acrylate-PEG.

In some embodiments, GGGGRGDSP peptide is covalently attached to acrylate-PEG, providing acrylate-PEG-GGGGRGDSP (APR).

In some embodiments, at least one of the one or more polymers are semi-synthetic.

In some embodiments, one or more hypoxia-inducing agents are covalently attached to at least one polymer of the one or more polymers.

In some embodiments, the covalent attachment of the one or more hypoxia-inducing agent to a polymer comprises a chemical moiety selected from the group consisting of —NHC(O)—, —NHC(O)CH₂O—, —C(O)O—, —NHC(O)O—, —CONHNHC(O)—, —NHC(O)NH—, —NHC(S)NH—, —SO₂—, —SO₂(CH₂CH₂)S—, —SC(O)O—, —NHCH₂CH₂C(O)O—, —SCH₂CH₂C(O)O—, —OCH₂CH₂C(O)O—, —NHCH₂CH₂SO₂—, —SCH₂CH₂SO₂—, —CH═N—, —CH═NO—, —CHN(OH)—, —N[CH₂CH(OH)CH₂O—]₂, —Si(O)—,—S—CH₂—, —S—C(CH₃)—, —NH—, —N—, —SS—,

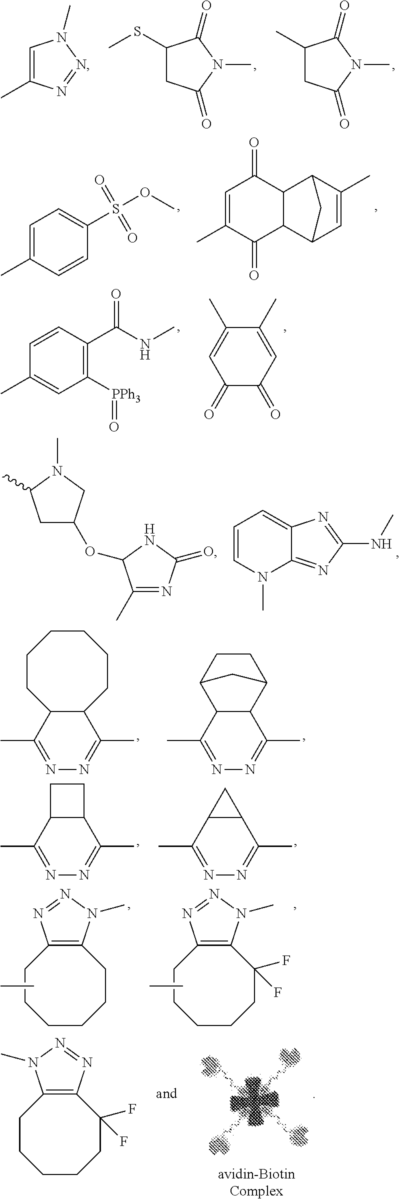

In some embodiments, the covalent attachment of the one or more hypoxia-inducing agent to comprises —NHC(O)—.

In some embodiments, the hypoxia-inducing agent is covalently attached to acrylate-PEG.

In some embodiments, at least one hypoxia-inducing agent is an enzyme.

In some embodiments, each hypoxia-inducing agent is an enzyme.

In some embodiments, at least one hypoxia-inducing agent is independently selected from the group consisting of oxidase, catalase (CAT), and ferulic acid.

In some embodiments, the hypoxia-inducing agent is an oxidase; and the oxidase is selected from the group consisting of glucose oxidase (GOX), galactose oxidase, pyranose 2-oxidase, NADPH oxidase, monoamine oxidase, and lactate oxidase.

In some embodiments, the cryogel comprises GOX.

In some embodiments, the cryogel comprises CAT.

In some embodiments, GOX is covalently attached to acrylate-PEG, providing acrylate-PEG-glucose oxidase (APG).

In some embodiments, CAT is covalently attached to acrylate-PEG, providing acrylate-PEG-catalase (APC).

In some embodiments, the cryogel comprises HAGM, APG, and APC.

In some embodiments, the cryogel comprises HAGM, APR, APG, and APC.

In some embodiments, the cryogel further comprises a lyoprotectant.

In some embodiments, the lyoprotectant is selected from the group consisting of trehalose, sucrose, glucose, lactose, mannose, fructose, galactose, maltose, sorbitol, mannitol, dextran, and polyvinylpyrrolidone.

In some embodiments, the cryogel further comprises a bioactive molecule.

In some embodiments, the bioactive molecule is selected from the group consisting of a lipid, a protein, or a nucleic acid.

In some embodiments, the bioactive molecule is selected from the group consisting of a cytokine, a chemokine, and a checkpoint inhibitor.

In some embodiments, the present disclosure relates to a hypoxia-inducing construct, comprising a cryogel and a support.

In some embodiments, the cryogel contacts the support.

In some embodiments, the support is selected from the group consisting of plate comprising a plurality of wells, a Petri dish, and a flask.

In some embodiments, the support is a plate comprising a plurality of wells.

In some embodiments, the present disclosure relates to a method of reducing concentration of oxygen in a medium, comprising contacting the medium with a hypoxia-inducing cryogel or a hypoxia-inducing construct.

In some embodiments, the medium comprises a cell culture medium.

In some embodiments, the medium comprises glucose, galactose, pyranose, NADPH, an amine, or lactate.

In some embodiments, the medium comprises glucose.

In some embodiments, the oxygen concentration is reduced by an amount from about 70% to about 99%. In some embodiments, the oxygen concentration is reduced by an amount from about 80% to about 99%. In some embodiments, the oxygen concentration is reduced by an amount from about 90% to about 99%. In some embodiments, the oxygen concentration is reduced by about 75%. In some embodiments, the oxygen concentration is reduced by about 95%. In some embodiments, the oxygen concentration is reduced by about 99%.

In some embodiments, the oxygen concentration is reduced by an amount from about 70% to about 99% within a period of time from about 1 min to about 30 min after the medium is contacted with a hypoxia-inducing cryogel.

In some embodiments, the oxygen concentration is reduced by an amount from about 70% to about 99% within a period of time from about 1 min to about 20 min after the medium is contacted with a hypoxia-inducing cryogel.

In some embodiments, the oxygen concentration is reduced by an amount from about 70% to about 99% within a period of time from about 1 min to about 10 min after the medium is contacted with a hypoxia-inducing cryogel.

In some embodiments, the oxygen concentration is reduced by an amount from about 70% to about 99% within about 1 min after the medium is contacted with a hypoxia-inducing cryogel.

In some embodiments, the oxygen concentration is maintained within a range from about 5 μM to about 50 μM for a period of time from about 48 h to about 264 h after the medium is contacted with a hypoxia-inducing cryogel.

In some embodiments, the medium comprises H₂O₂, and the concentration of H₂O₂ is less than about 10 μM. For example, the concentration of H₂O₂ is less than about 10 μM, less than about 9 μM, less than about 8 μM, less than about 7 μM, less than about 6 μM, less than about 5 μM, less than about 4 μM, less than about 3 μM, less than about 2 μM, less than about 1 μM, less than about 0.5 μM, or less than about 0.1 μM.

In some embodiments, the medium comprises H₂O₂, and the concentration of H₂O₂ is less than about 1 μM. In some embodiments, the medium comprises H₂O₂, and the concentration of H₂O₂ is less than about 0.1 μM.

In some embodiments, the medium does not comprise H₂O₂.

In some embodiments, the present disclosure relates to a method of inducing hypoxia, comprising contacting a cell with a medium, wherein the medium comprises a hypoxia-inducing cryogel or a hypoxia-inducing construct.

In some embodiments, the medium is a cell culture medium.

In some embodiments, the present disclosure relates to a method of inducing hypoxia comprising contacting a cell with a hypoxia-inducing cryogel or a hypoxia-inducing construct.

### EXAMPLES

Materials were obtained as follows:

| Material Description | Commercial Source |
|---|---|
| Hyaluronic Acid Salt | Millipore Sigma |
| Glycidyl Methacrylate | Millipore Sigma |
| Phosphate Buffer Saline (PBS) | Millipore Sigma |
| Dimethylformamide | Millipore Sigma |
| Triethylamine | Millipore Sigma |
| Acetone | Millipore Sigma |
| Acrylate-PEG-N-hydroxysuccinimide | JenKem Technology |
| GGGGRGDSP (peptide) | Peptide2.0 |
| Catalase | Millipore Sigma |
| Glucose Oxidase | Millipore Sigma |
| NHS-Rhodamine | Millipore Sigma |

Hyaluronic acid (HA) was conjugated with glycidyl methacrylate (GM) as followed: HA salt (5 g) was dissolved in PBS (1 L, pH 7.4) and mixed with dimethylformamide (DMF, 335 mL), GM (62 mL), and triethylamine (TEA, 46 mL). The reaction was allowed to proceed for ten days at room temperature (RT) and the mixture was precipitated in a large excess of acetone, filtered using grade 4 Whatman paper, and dried in a vacuum oven overnight at RT. The resulting product, hyaluronic acid glycidyl methacrylate (HAGM), was characterized by ¹H NMR.

Acrylate-PEG-GGGGRGDSP (APR) was synthesized by coupling the amine-terminated GGGGRDGSP peptide to acrylate-PEG-N-hydroxysuccinimide (molar ratio, 1:1). Briefly, acrylate-PEG-N-hydroxysuccinimide (100 mg) and GGGGRDGSP peptide (22.3 mg) were mixed in 1 m NaHCO₃ buffer solution at pH 8.5, allowed to react for 4 hours at RT, and freeze-dried overnight. Similarly, acrylate-PEG-catalase (APC) and acrylate-PEG-glucose oxidase (APG) were synthesized by coupling the enzymes to acrylate-PEG-N-hydroxysuccinimide comonomers (molar ratio 1:3).

### Example 1

### Fabrication Process of HA-Based HIC_{2D}

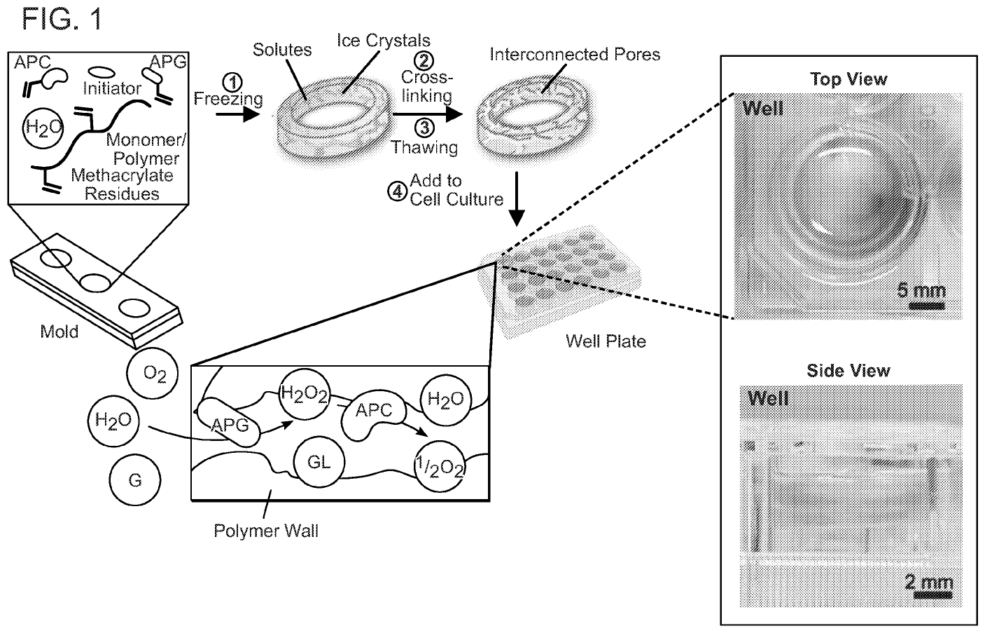

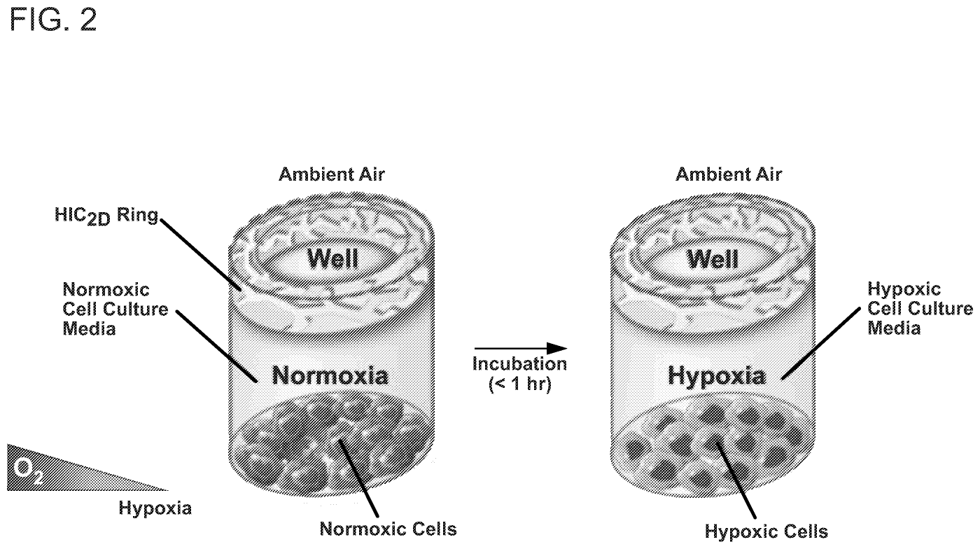

Hypoxia-inducing cryogels device for 2D hypoxic cell culture (HIC_{2D}) were fabricated with 4% hyaluronic acid glycidyl methacrylate (HAGM), 0.1% (w/v) acrylate-PEG-glucose oxidase (APG), and 1.5% acrylate-PEG-catalase (APC). HIC_{2D} were fabricated via cryopolymerization at −20° C. through a free radical cross-linking mechanism using tetramethylethylenediamine (0.14% v/v) and ammonium persulfate (0.58% v/v) as the initiator system. After complete polymerization, HICs_{2D} were allowed to thaw at room temperature to melt ice crystals (i.e., porogens) (FIG. 1). HICs_{2D} were then washed with phosphate buffered saline (PBS) to remove unreacted precursors, 70% ethanol for sanitization and finally two PBS washes for ethanol removal. Two-dimensional HICs_{2D} (volume: ˜200 μL) in the shape of rings (i.e., hollow centers) were prepared for use in 24-well plates. When inserted into a cell culture media-containing well, HICs_{2D} deplete oxygen rapidly (<1 h), create a hypoxic environment, and induce cellular hypoxia (FIG. 2).

### Example 2

### Fabrication Process of HA-Based HICs

HICs were fabricated with 4% hyaluronic acid glycidyl methacrylate (HAGM), 0.8% Acrylate-PEG-GGGGRGDSP (APR), 0.1% Acrylate-PEG-Glucose oxidase (APG), and 1% Acrylate-PEG-Catalase (APC) (w/v). HICs were fabricated via cryopolymerization at −20° C. through a free radical cross-linking mechanism using tetramethylethylenediamine (0.14% v/v) and ammonium persulfate (0.58% v/v) as the initiator system. After complete polymerization, HICs were allowed to thaw at room temperature to melt ice crystals (i.e., porogens) (FIG. 1). HICs were then washed with distilled H₂O to remove unreacted precursors,

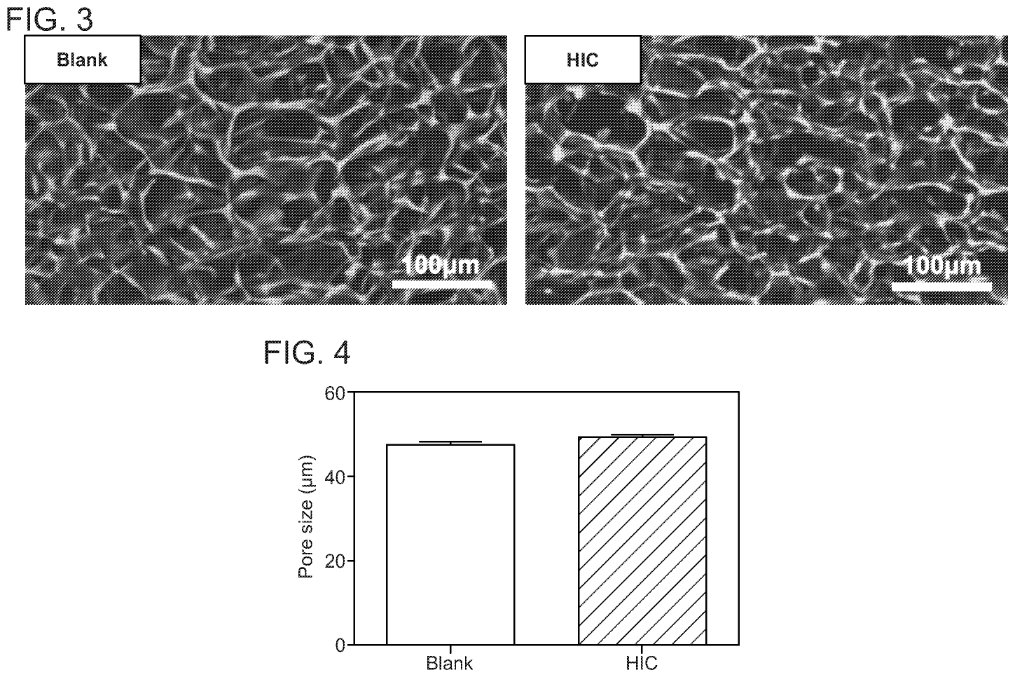

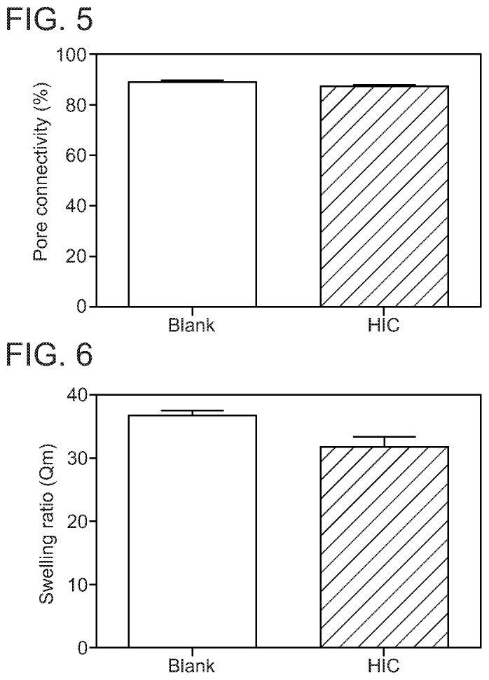

A critical property of cryogel-based biomaterials relies on their ability to produce a system of interconnected macropores. Here, the macrostructure of HICs was imaged by confocal microscopy and compared to blank cryogels (without APG and APC). As shown in FIGS. 3-5, HICs displayed highly interconnected pores (˜85%), with sizes of 49±2 μm. No differences were observed when compared to the porous structure of regular HAGM cryogels (blanks).

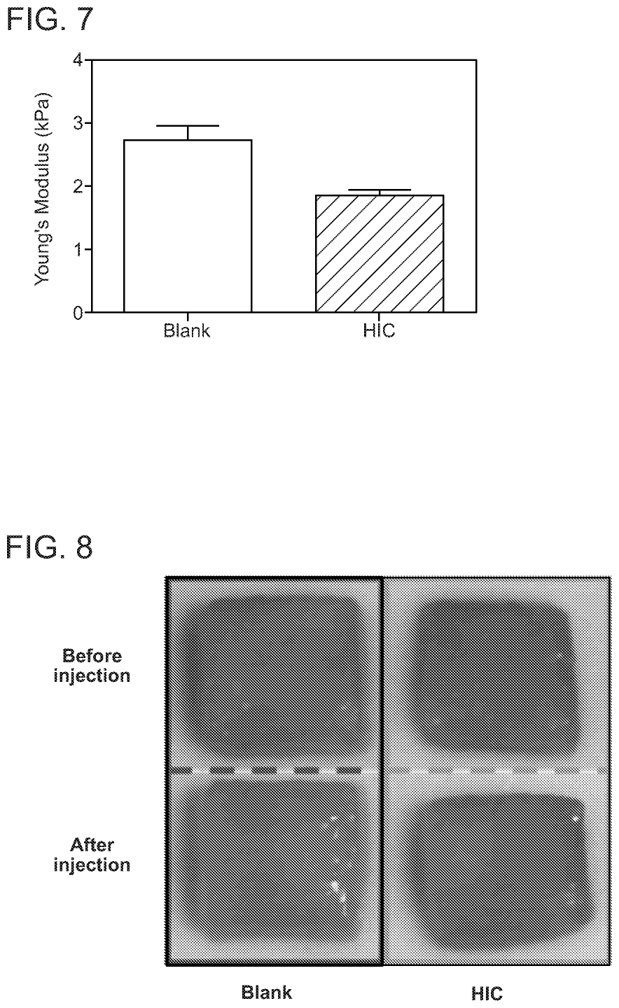

Next, the influence of the network microstructure of HICs on their mechanical properties was evaluated (FIGS. 6-8). Although the swelling ratio of HICs was similar to that of blank cryogels (Qm˜30), a slight decrease of Young's modulus was observed (1.8±0.05 kPa for HICs vs 2.8±0.22 kPa for blank cryogels). Consequently, HICs appeared to be slightly weaker than blank cryogels but retained their capacity to be injected through small 16-gauge needles without any physical damage. Taken together, these results show that HICs are highly macroporous hydrogels with shape memory properties which allows them to be syringe injected, and suitable for biological assays due to their high swelling ratio as well scaffolds for tissue engineering applications due to their Young's moduli comparable to soft native tissues.

### Example 3

### Oxygen Depletion by HIC_{2D} for 2D Cell Culture

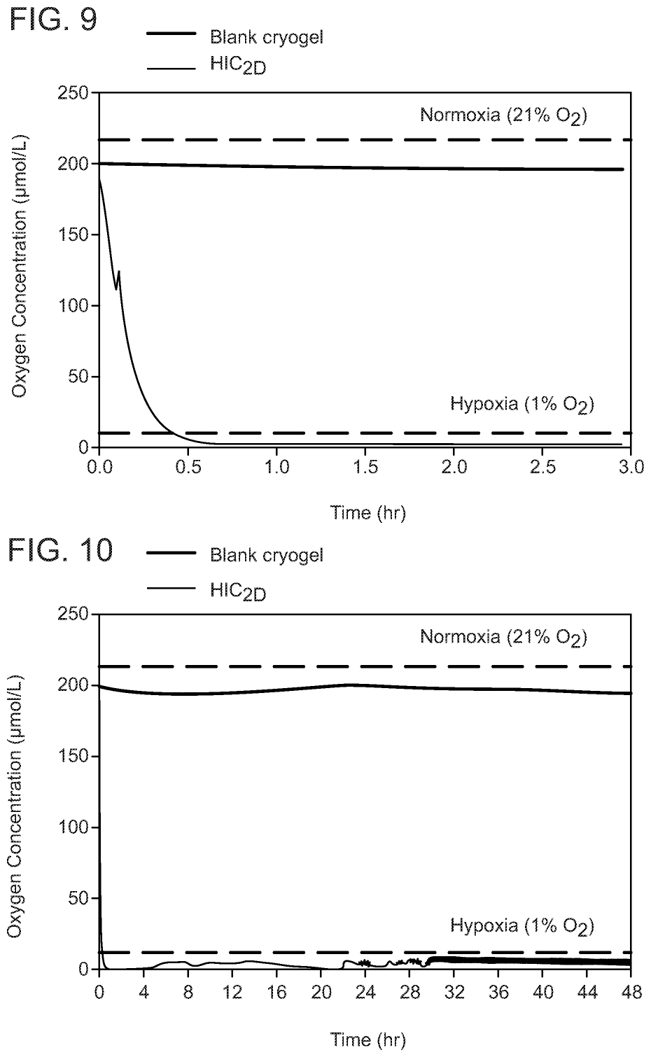

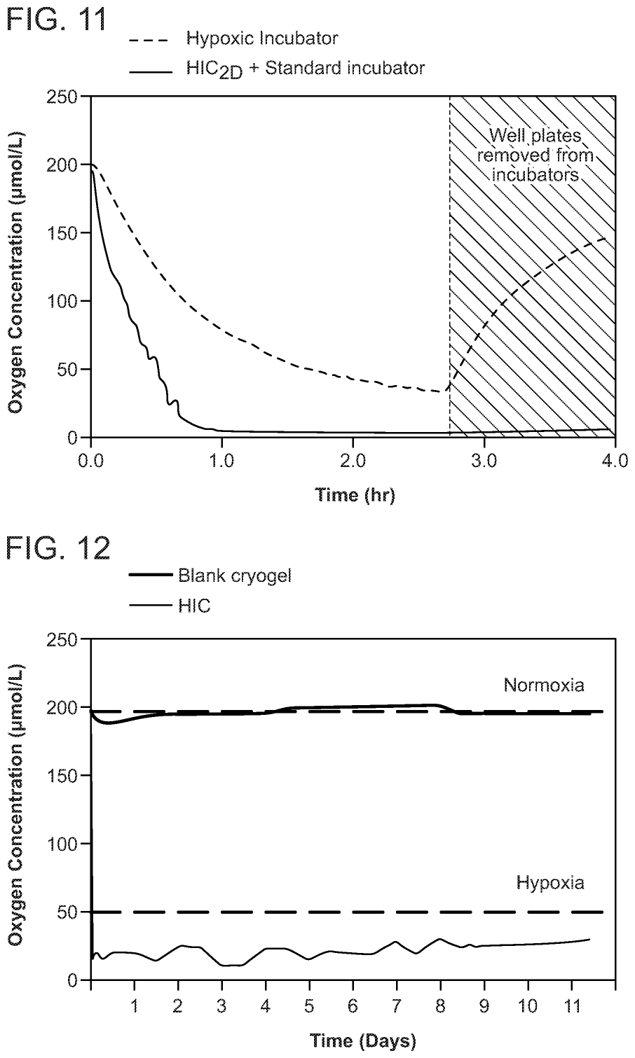

The oxygen depletion kinetics by HICs in 24-well plates was examined. HICs or blank cryogels were added to wells containing media. Needle-type oxygen probes were positioned in the farthest point away from HICs2D, and the media's dissolved oxygen concentration was measured every 5 min for 48 h in normoxia (21% O₂). HICs_{2D} induced a dramatic reduction of oxygen concentration from 200 μmol/L (21% O₂ in media) to 5 μmol/L (˜1% O₂ in media) in approximately 25 minutes (FIG. 9) and maintain hypoxia for 48 h (FIG. 10). As expected, wells with blank cryogels were normoxic for the duration of the experiment. Next, the oxygen depletion kinetics of media by HICs_{2D} and a tri-gas incubator, a conventional hypoxic cell culture incubator (Thermo Napco CO₂ 1000 hypoxic incubator) were compared. HICs_{2D} rapidly induced hypoxia (˜5% O₂) within 30 minutes, whereas it took the incubator>100 minutes to induce hypoxia (FIG. 11). Furthermore, the hypoxic environment induced by the hypoxic incubator was not stable over time. For instance, once hypoxia was reached (<5% O₂), the well plates were removed from both incubators (standard and hypoxic) to examine hypoxia maintenance and the rate of equilibration to normoxia when stored under atmospheric oxygen. The well plate rapidly lost hypoxia (<5 minutes), showing the limitation of hypoxic incubators.

### Example 4

### Oxygen Depletion by HICs

The chemically modified glucose oxidase (APG) can catalyze the oxidation of glucose into hydrogen peroxide (H₂O₂) and D-glucono-δ-lactone. In addition, the chemically modified catalase (APC) can increase the rate of H₂O₂ degradation into oxygen and water to suppress any unwanted toxic side reactions.

Glucose+H₂O+O₂O+O₂ [symbol] D-glucono-δ-lactone+H₂O₂

H₂O₂ [symbol] 1/₂ O₂+H₂O

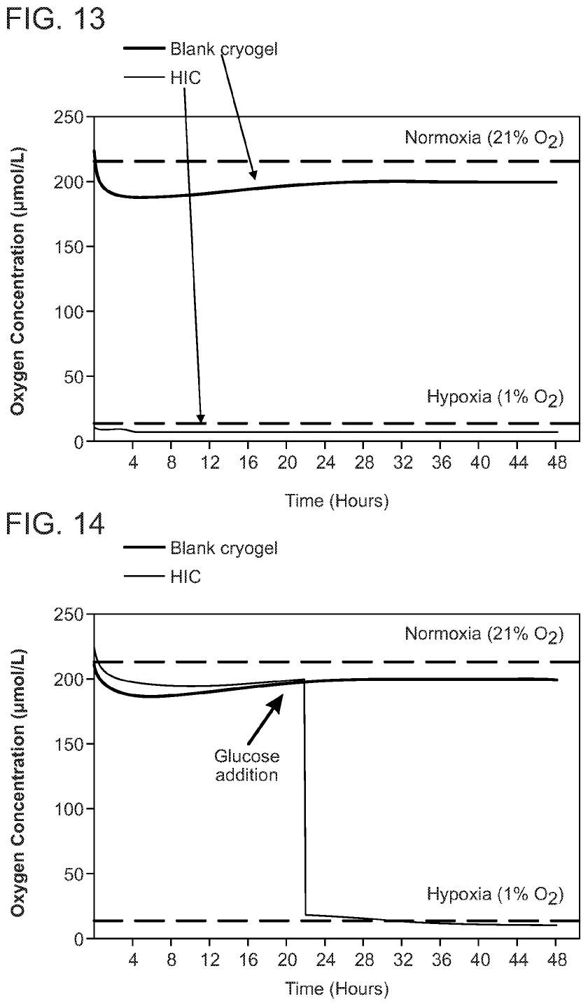

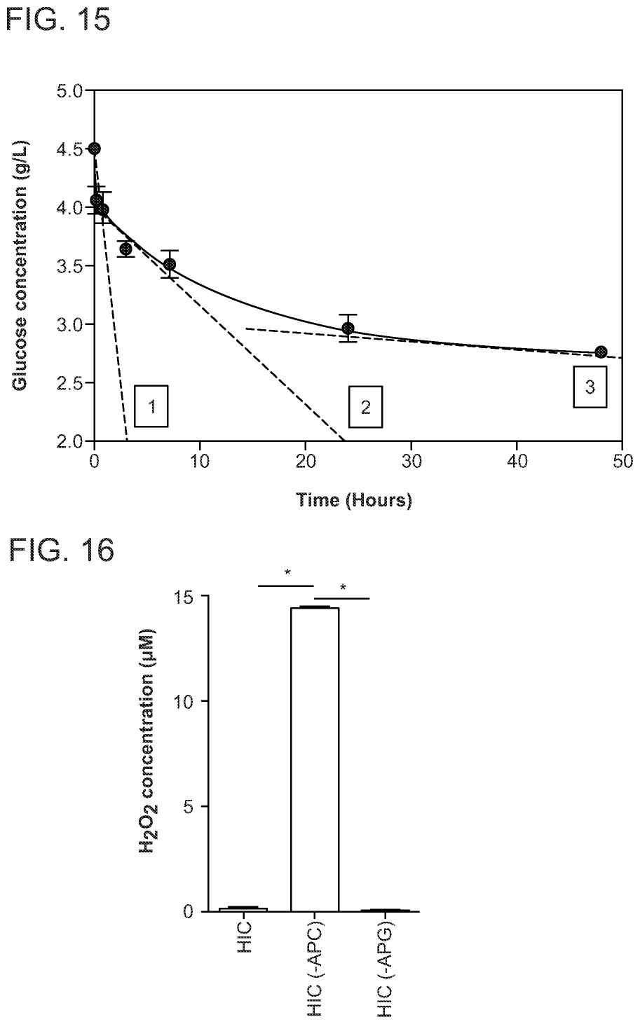

To determine the oxygen depletion rates of HICs, needle-type oxygen probes were used, positioned in the center of each HICs, and the media's dissolved concentration of oxygen was measured every 5 min for 11 days (FIG. 12). Blank cryogels were used as a control of normal oxygen concentration. In normoxia, HICs induced a dramatic reduction of the oxygen concentration from 200 μmol/L (˜21% of oxygen in media) to 10 μmol/L (˜1% of oxygen in media) in only a few minutes (FIG. 13). Moreover, this oxygen depletion is dependent on the presence of glucose (FIG. 14). In a glucose-free medium, HICs did not induce hypoxia, and normoxia was maintained throughout the duration of the experiment. However, when glucose was added (4.5 g/L), HICs depleted oxygen quickly (˜10 min) and reached hypoxia (˜1 O₂%). As expected, oxygen concentration remained unchanged with blank cryogels. The kinetics of HIC-mediated glucose consumption has also been investigated (FIG. 15). During the oxygen depletion period, a complex set of processes including the initial oxygen depletion (1), neo-dissolution of oxygen from air to the media (2) and the equilibrium of HIC-induced oxygen depletion (3) took place. At this point glucose consumption decreased to 0.2 g/L of glucose per day.

Finally, the capacity of HICs to prevent the formation of toxic H₂O₂, a byproduct generated during the enzymatic oxygen depletion (FIG. 16), was investigated. HICs resulted in production of a negligible level of H₂O₂ (˜0.1 μM), below the level of toxicity (10 μM). However, use of APC-free HICs led to an increased level of H₂O₂ (˜14 μM), above the toxicity levels, thus suggesting the need of incorporating catalase into HICs. As expected, APG-free HICs did not generate H₂O₂ as glucose oxidase is required (negative control).

### Example 5

### HIC_{2D}-Mediated Cellular Hypoxia

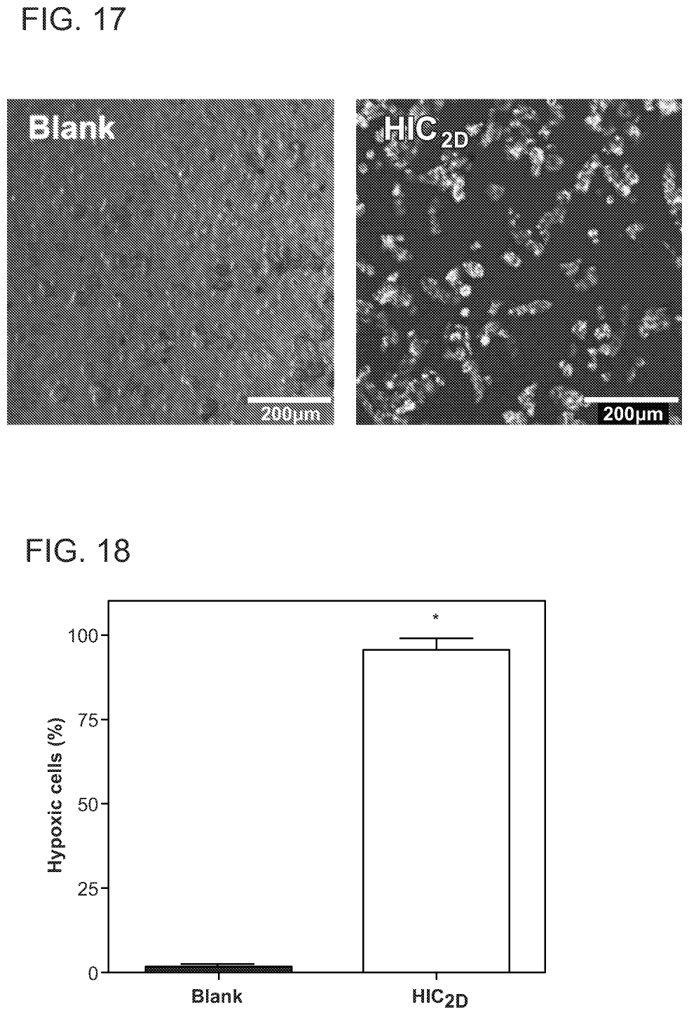

The ability of HIC_{2D} to induce cellular hypoxia in B16-F10 melanoma cells was next examined. HIC_{2D} rings or blank cryogel rings were added to wells containing hypoxia-stained (e.g., Image-iT® Red) B16-F10 melanoma cells in DMEM media supplemented with 7.5 g/L of D-glucose (FIG. 2). After 18 h of cell culture with HICs_{2D} or blank cryogels, cellular hypoxia was evaluated qualitatively (FIG. 17) and quantitatively (FIG. 18) by confocal microscopy and data processing. The results indicate that B16-F10 melanoma cells cultured in wells containing a HIC_{2D} were highly hypoxic (>95%), whereas cells cultured with blank cryogels were not (<1%).

### Example 6

### Cytocompatibility and HIC-Mediated Cellular Hypoxia

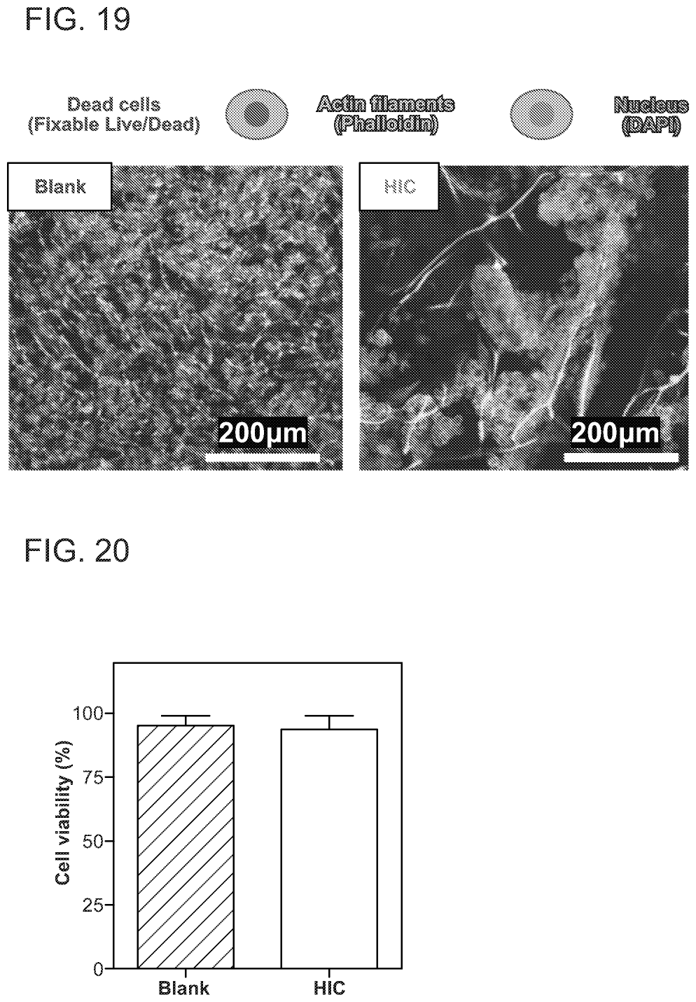

Cell viability within HICs as an indication of their cytocompatibility was examined. HICs were partially dehydrated, then seeded with 1×10⁵ B16-F10 melanoma cells and incubated at 37° C. for 24 h in normoxia (FIGS. 19-20). In HICs, B16-F10 cells had a high viability of 95%±4%, comparable to blank cryogels (97%±3%). This observation can be correlated to the absence of H₂O₂ release from HICs. More surprisingly, cells cultured within HICs spontaneously reorganized in organoid-like 3D structures with strong cell-cell interactions. In contrast, cells cultured within blank gels only interacted with the polymer walls, resulting in the formation of a monolayer of stretched cells within the 3D scaffolds. Regardless of the composition, cells homogeneously attached and spread throughout the constructs. These results indicate that the developed HICs display the necessary cytocompatibility and promote cell adhesion, viability, and reorganization into organoid-like structures.

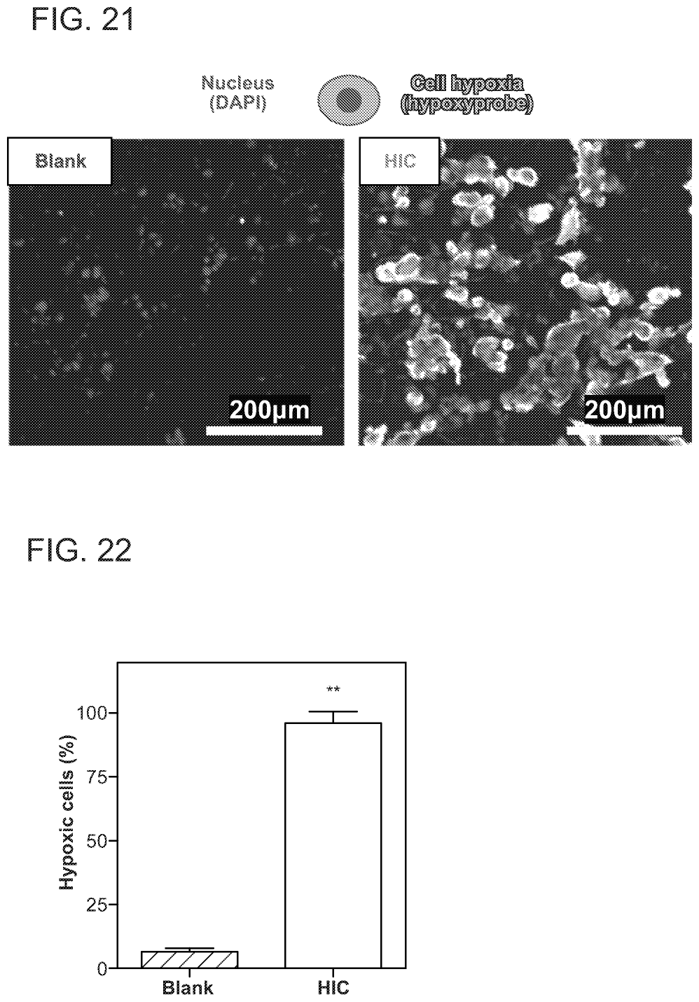

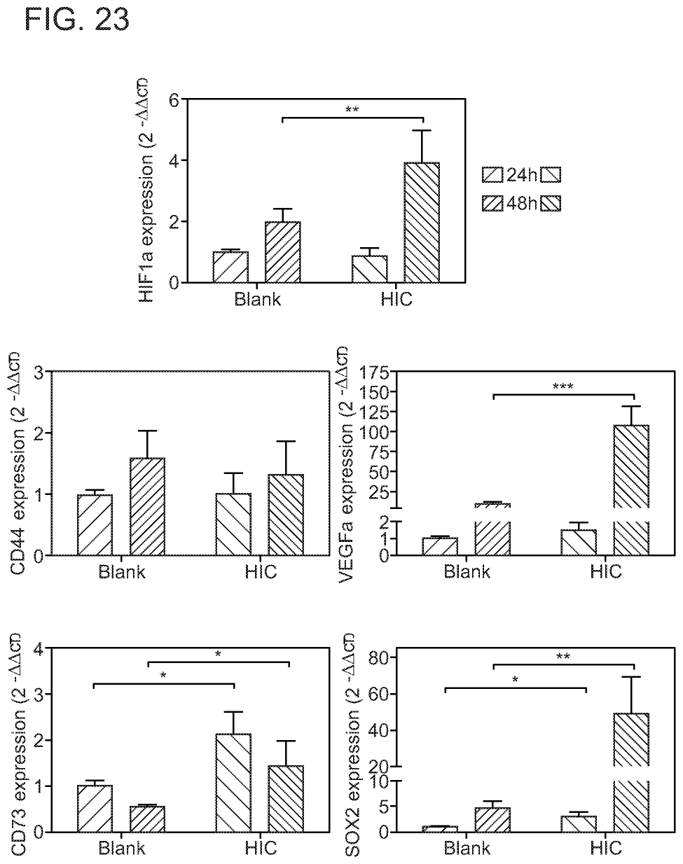

Cellular hypoxia was assessed in normoxia by analyzing the number of hypoxic B16-F10 cells within HICs and compared to blank cryogels (FIGS. 21, 22). After 24 h incubation, 96.5±2% of cells were hypoxic within HICs. In contrast, only 6.5±1.1% of cells were hypoxic in blank cryogels. Taken together, these results validate the capability of HICs to induce hypoxic conditions at a cellular level. Additionally, with their high cytocompatibility, HICs seem to be suitable for tissue modeling or in vitro studies to assess the impact of hypoxia on cells or biological processes.

### Example 7

### HICs-Induced Switch in Cancer Cell Phenotypes

The impact of HICs on the gene expression profile of 4T1 breast cancer cells was investigated. The cancer cells were cultured for 24 h or 48 h in normoxia within HICs or blank cryogels prior to mRNA extraction and qPCR analysis (FIG. 23). After 24 h 4T1 cells within blank cryogels had low-level expression of HIF1a (hypoxia-inducible transcription factor), VEGFa (angiogenesis marker), CD44 and SOX2 (cancer stemness markers), as well as CD73 (receptor responsible for tumor-mediated immunosuppression in hypoxia). Expression of these markers slightly increased after 48 h, mainly due to cell confluency, but also the gradient of oxygen present within cryogels (cell metabolism and oxygen diffusion). After 24 h incubation in HICs the expression of CD73 and SOX2 was significantly higher as compared to blank cryogels. More strikingly after 48 h, not only CD73 and SOX2 but also VEGFa and HIF1a expression level dramatically increased within HICs. Surprisingly, no change regarding CD44 expression has been observed. The present blank cryogels and HICs are composed of HA, which is a natural ligand of CD44 receptors, and it can be hypothesized that the maximum expression of CD44 is already reached due to the constructs' composition. Altogether, these data indicate that HICs are able to switch the phenotype of cancer cells and trigger cancer stemness, immunosuppression, and neovascularization, therefore mimicking the phenotype of native aggressive tumors found in vivo.

### Example 8

### HICs Induce Cancer Cell Resistance to Chemotherapeutic Agents

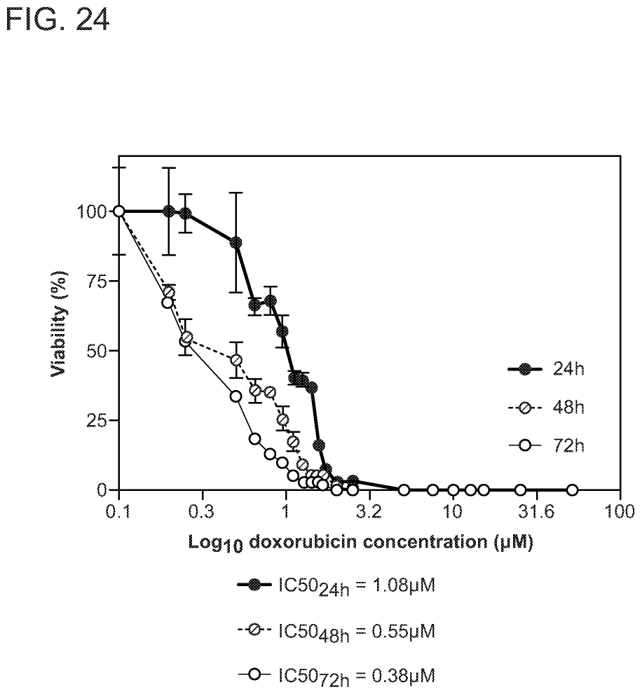

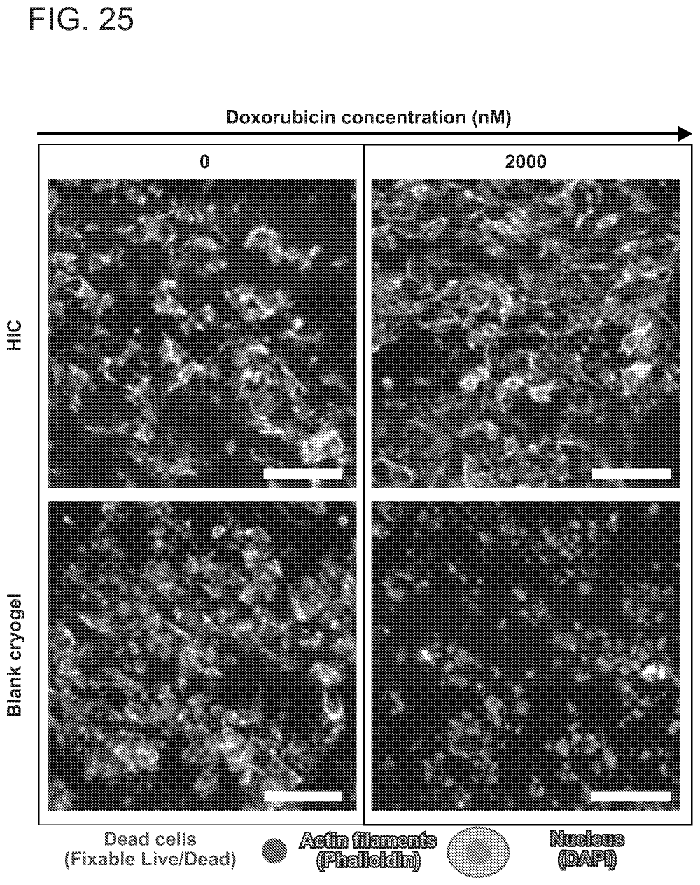

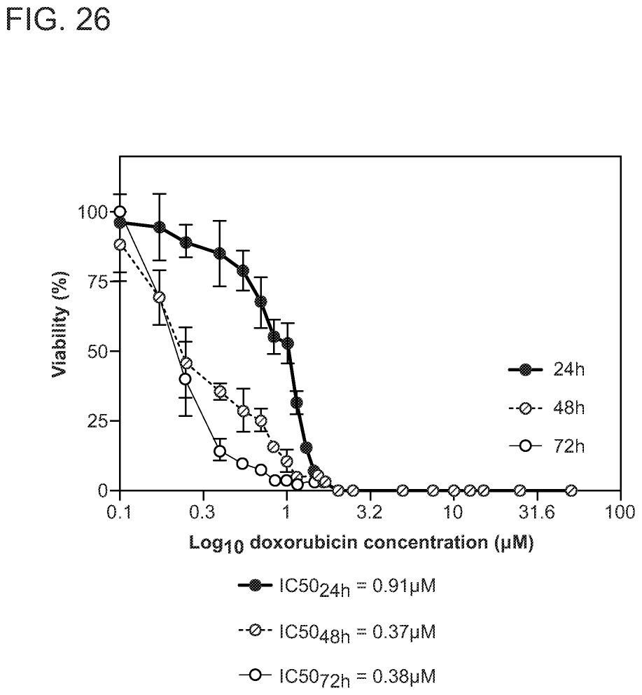

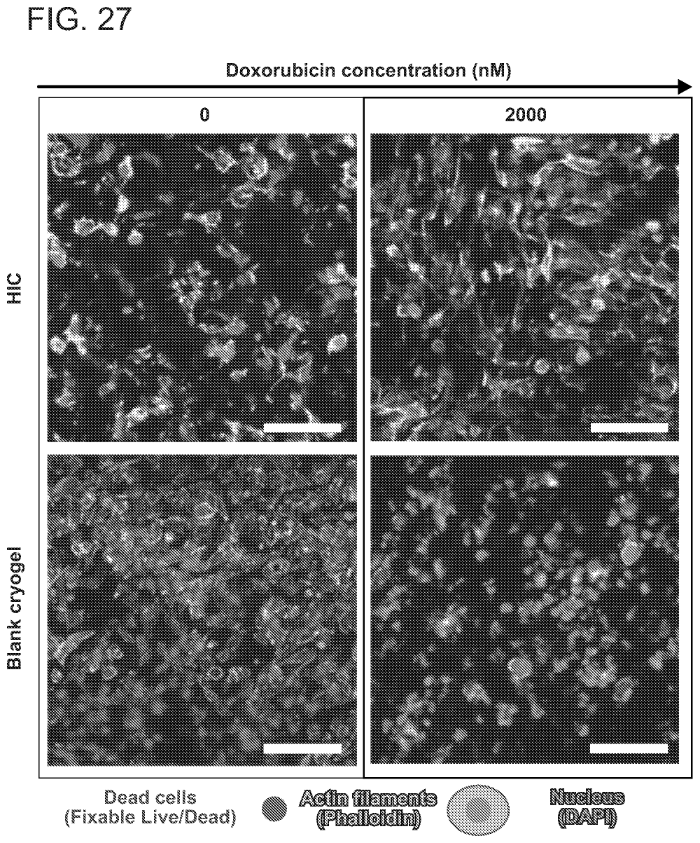

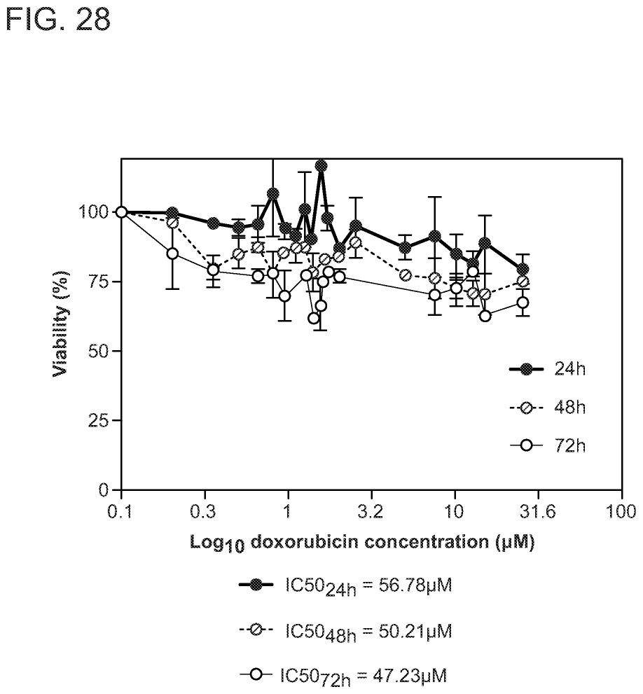

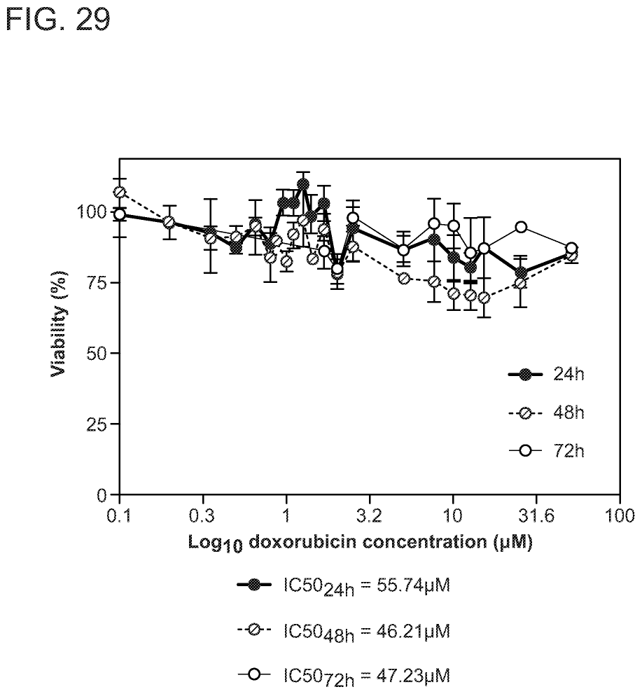

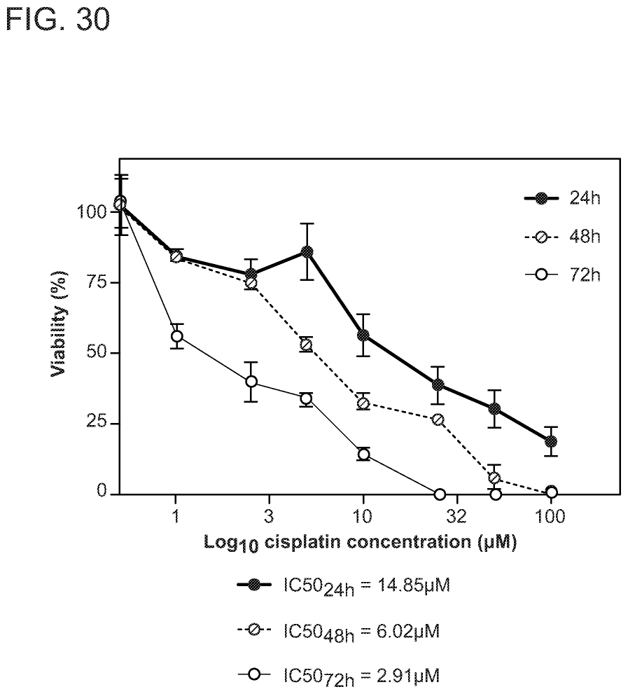

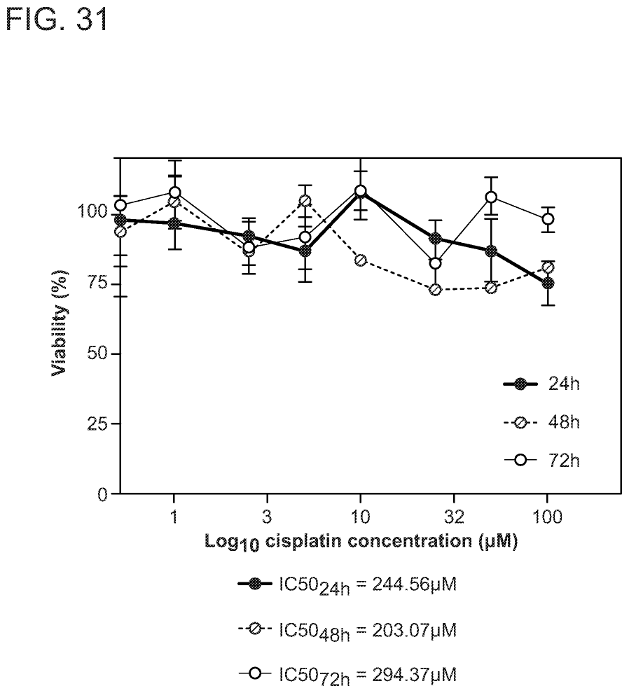

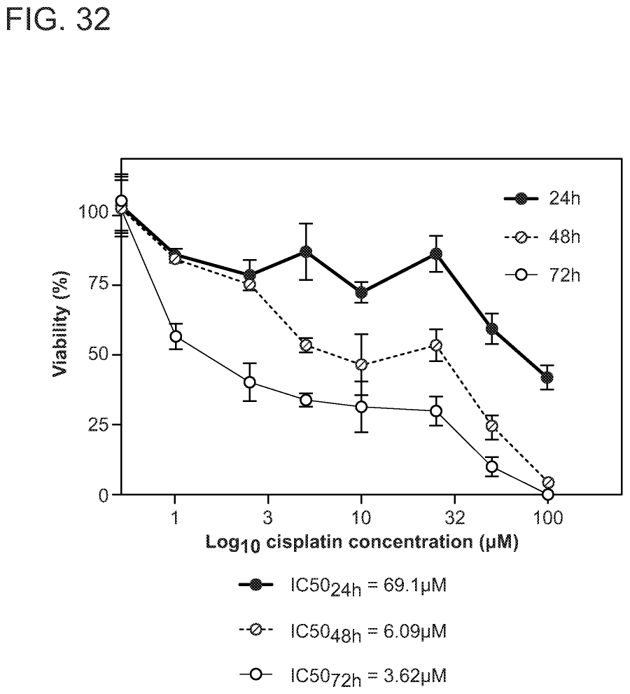

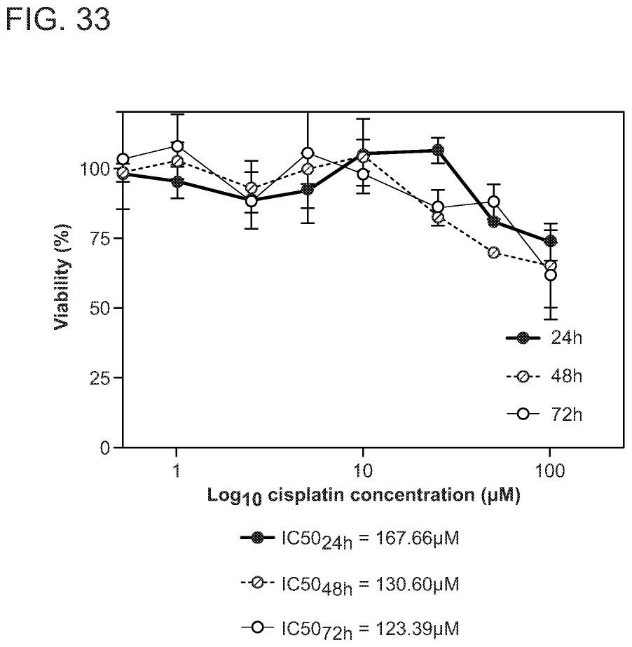

The capacity of HICs to prevent cancer cell death when exposed to chemotherapeutic drugs was analyzed. 4T1 breast cancer cells or B16-F10 melanoma cells were cultured in normoxia within blank cryogels or HICs and treated with several concentrations of doxorubicin or cisplatin (0.1-100 μM) for 24 h, 48 h, or 72 h. Both 4T1 (FIGS. 24 and 25) and B16-F10 (FIGS. 26 and 27) cells within blank cryogels were sensitive to doxorubicin treatment. IC50 of 1.08 μM, 0.55 μM and 0.33 μM for 4T1 cells and 0.91 μM, 0.37 μM and 0.38 μM for B16-F10 cells were observed after 24 h, 48 h and 72 h treatment respectively. However, cells cultured in HICs showed a dramatic increase in their resistance to doxorubicin. 4T1 cells (FIGS. 25 and 28) in HICs displayed an IC50 of 56.78 μM, 50.71 μM and 47.23 μM after 24 h, 48 h and 72 h treatment respectively, 50-fold to 150-fold higher as compared to the blank cryogels. In the case of B16-F10 cells, a 60-fold to 150-fold increase of resistance to doxorubicin has also been observed, with IC50 values of 55.74 μM, 46.21 μM and 47.23 μM after 24 h, 48 h and 72 h treatment (FIGS. 27 and 29). Similar results were observed with cisplatin treatments of 4T1 and B16-F10 cells (FIGS. 30-33). HICs induced up to 100-fold increase in cancer cell resistance to cisplatin after 72 h compared to blank cryogels. Altogether, these results clearly demonstrate that HICs induce chemotherapeutic resistance to cancer cells and can be used as a drug screening platform to mimic in vivo acquired resistance of tumors to drugs due to local hypoxia.

### INCORPORATION BY REFERENCE

All U.S. patents and U.S. and PCT patent publications mentioned herein are hereby incorporated by reference in their entirety as if each individual patent or publication was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A hypoxia-inducing cryogel, comprising one or more polymers; and one or more hypoxia-inducing agents.

2. The cryogel of claim 1, wherein the one or more polymers are biocompatible and/or hydrophilic.

3. (canceled)

4. The cryogel of claim 1, wherein the one or more polymers are independently selected from the group consisting of a DNA strand, a peptide, a protein, alginate, hyaluronic acid, chitosan, heparin, carboxymethyl cellulose, cellulose, carob gum, hyaluronic acid glycidyl methacrylate (HAGM), methacrylated gelatin, methacrylated alginate, poly(ethylene glycol) (PEG), acrylate-PEG, methacrylate-PEG, PEG-co-poly(glycolic acid), PEG-co-poly(L-lactide), poly(2-hydroxyethyl methacrylate) (pHEMA), poly-2-hydroxyethylacrylate (polyHEA), polyacrylamide (PAAm), and poly(N-isopropylacrylamide) (PNIPAAm), and copolymers and combinations thereof.

5.-7. (canceled)

8. The cryogel of claim 4, wherein the peptide is selected from the group consisting of GRGDS, GGGGRGDSP, and GFOGER.

9. The cryogel of claim 4, wherein the peptide or the protein is covalently attached to at least one polymer of the one or more polymers.

10. (canceled)

11. The cryogel of claim 9, wherein GGGGRGDSP peptide is covalently attached to acrylate-PEG, providing acrylate-PEG-GGGGRGDSP (APR).

12. (canceled)

13. The cryogel of claim 1, wherein the one or more hypoxia-inducing agents are covalently attached to at least one polymer of the one or more polymers.

14.-16. (canceled)

17. The cryogel of claim 1, wherein at least one hypoxia-inducing agent is an enzyme.

18. (canceled)

19. The cryogel of claim 1, wherein at least one hypoxia-inducing agent is independently selected from the group consisting of oxidase, catalase (CAT), and ferulic acid.

20. The cryogel of claim 19, wherein the hypoxia-inducing agent is an oxidase;
and the oxidase is selected from the group consisting of glucose oxidase (GOX), galactose oxidase, pyranose 2-oxidase, NADPH oxidase, monoamine oxidase, and lactate oxidase.

21.-22. (canceled)

23. The cryogel of claim 19, wherein the at least one hypoxia-inducing agent is
(i) an oxidase, wherein the oxidase is GOX covalently attached to acrylate-PEG (APG); or
(ii) CAT covalently attached to acrylate-PEG (APC).

24-25. (canceled)

26. The cryogel of claim 1, comprising HAGM, APR, APG, and APC.

27.-28. (canceled)

29. The cryogel of claim 1, further comprising a bioactive molecule.

30. (canceled)

31. The cryogel of claim 29, wherein the bioactive molecule is selected from the group consisting of a cytokine, a chemokine, and a checkpoint inhibitor.

32.-35. (canceled)

36. A method of reducing the concentration of oxygen in a medium, comprising contacting the medium with a hypoxia-inducing cryogel of claim 1.

37.-39. (canceled)

40. The method of claim 36, wherein the oxygen concentration is reduced by an amount from about 70% to about 99% from about 80% to about 99%, or from about 90% to about 99%.

41.-42. (canceled)

43. The method of claim 36, wherein the oxygen concentration is reduced by at least about 95%, or by at least about 75%.

44. (canceled)

45. The method of claim 36, wherein the oxygen concentration is reduced by an amount from about 70% to about 99% within a period of time from about 1 min to about 30 min, from about 1 min to about 20 min, from about 1 min to about 10 min, or within about 1 min after the medium is contacted with a hypoxia-inducing cryogel.

46.-49. (canceled)

50. The method claim 36, wherein the medium comprises H₂O₂, and wherein the concentration of H₂O₂ is less than about 10 μm, less than about 1 μM, or less than about 0.1 μM.

51.-55. (canceled)

56. A method of inducing hypoxia in a cell, comprising contacting the cell with a hypoxia-inducing cryogel of claim 1.
